(19) United States
(12) Patent Application Publication
(10) Pub. No.: US 2021/0059343 A1
(43) Pub. Date: Mar. 4, 2021

(54) Medical Support Headwear

(71) Applicant: CO-AMPLIFY, LLC, Portland, ME (US)

(72) Inventor: Meghan Carey, Portland, ME (US)

(73) Assignee: CO-AMPLIFY, LLC, Portland, ME (US)

(21) Appl. No.: 17/010,253

(22) Filed: Sep. 2, 2020

Related U.S. Application Data
(60) Provisional application No. 62/895,066, filed on Sep. 3, 2019.

(51) Int. Cl. A42B 1/24; A61N 1/36; A42B 7/00; A42B 1/06

(52) U.S. Cl. CPC A42B 1/241; A61N 1/36038; A42B 1/062; A42B 1/066; A42B 7/00

(57) ABSTRACT

Disclosed herein is a novel medical support headwear device and method of use for ensuring proper placement and connectivity of medical devices. The medical support headwear device includes one or more side panels, each side panel including an internal layer attached to an external layer to form a pocket therein, and an access point formed in each of the side panels to provide access to the pocket.

### BACKGROUND

### 1. Field of the Invention

The present inventive subject matter relates to the field of apparel manufacturing and products made therefrom. More specifically, the present inventive subject matter relates to medical device support clothing within the field.

### 2. Discussion of Background Information

Decades of research have proven that deaf children can develop speech and language at typical rates if they receive cochlear implants very early in life. As of 2020, the target age for a new pediatric implant is as young as 6-9 months.

Cochlear implants are wearable hearing devices that can restore hearing in people with severe hearing loss by bypassing damaged portions of the ear to deliver sound signals to the auditory nerve. Cochlear implants include both internal and external components, where the internal component is an implanted receiver that functions with three core external components: a microphone, a speech processor, and a transmitter.

The external components of a cochlear implant are typically arranged in one of three formats:
1) An over-the-ear speech processor and microphone with a short cable to the magnetically attached transmitter;
2) A clip-on speech processor with a longer cable to the microphone and magnetically attached transmitter; or
3) An all-in-one speech processor, microphone, and magnetically attached transmitter.

A magnet connects the external transmitter through the skin to the implanted receiver, which stimulates the auditory nerve with electrodes that have been surgically threaded into the cochlea.

Because typically developing pediatric patients have not yet acquired the language and communication skills necessary to articulate discomfort, the transmitter magnets are intentionally weak to prevent injury at the contact point. However, due to the weakness of the magnets, the transmitters fall off frequently, breaking the magnetic connection, and terminating access to sound. The weakness of the magnets becomes particularly problematic when a pediatric patient needs to wear headwear, such as a baseball hat, sun hat, winter hat or helmet because it is impractical to attach and secure the transmitter and then put on a piece of headwear without breaking the magnetic connection. Indeed, as the technology around wearable medical devices has advanced at an unprecedented rate, the landscape for medical support clothing has not kept up. Specifically, there are currently no headwear solutions with pocket modifications to ensure placement and connectivity of medical devices. As a result, guardians of pediatric patients are forced to choose between the child's auditory access and his or her safety, comfort, and warmth.

While headwear devices that ensure placement and connectivity of medical devices exist, existing devices fail to address the needs of cochlear implant recipients. For example, U.S. Patent Application US 2015/0223539 to Marco discloses an example of a headwear device for ensuring placement and connectivity of medical devices. The Marco device is a hat for securing external hardware of a cochlear implant or traditional hearing aid of a wearer, which comprises a body of conforming material to cover at least a portion of the wearer's head, an opposing pair of substantially wide under-chin straps, and a soft, conforming open mesh to cover the external hardware of the cochlear implant and provide for securing of the cochlear implant when worn by the wearer. However, the Marco device lacks pockets or other structure to secure the external transmitter. Instead, the Marco hat surrounds the external transmitter and functions as a shield, attempting to limit the child's ability to remove the external component while permitting an adult to inspect and, if necessary, reposition a dislodged external transmitter. Additionally, by utilizing an open mesh to cover the external hardware of the cochlear implant, the Marco device is incapable of shielding the external transmitter and the wearer from inclement weather conditions.

While other headwear solutions have been proposed to ensure placement and connectivity of medical devices, none of these inventions, taken either singly or in combination, adequately address or resolve the aforementioned problems. Therefore, a need exists for headwear solutions with pocket modifications to ensure placement and connectivity of medical devices.

### SUMMARY OF THE INVENTION

The present invention solves the problems associated with headwear devices that ensure placement and connectivity of medical devices.

The present invention is directed to a medical support headwear device. The medical support headwear device comprises one or more side panels; each of the one or more side panels comprising an internal layer attached to an external layer to form a pocket therein; and an access point formed in each of the one or more side panels to provide access to the pocket formed therein.

The present invention is also directed to a method for wearing medical support headwear comprising a first step of providing a medical support headwear device. The medical support headwear device comprises one or more side panels; each of the one or more side panels comprising an internal layer attached to an external layer to form a pocket therein; and an access point formed in each of the one or more side panels to provide access to the pocket formed therein. A second step includes placing the medical support headwear device on the user's head. A third step includes opening the access point formed in a first of the one or more side panels by separating the external layer from the internal layer to create an open access point. A fourth step includes inserting a CI transmitter through the open access point and into the pocket such that the CI transmitter lies in the pocket and in magnetic adhesion with the corresponding internal receiver components of the cochlear implant. A fifth step includes closing the open access point such that the CI transmitter is ensconced in the pocket.

### BRIEF DESCRIPTION OF THE DRAWINGS

These and other features, aspects and advantages of the present invention will become better understood with reference to the following description, appended claims, and accompanying drawings where:

FIG. 1 is a side view of a Bomber Style Headwear embodiment of the present invention.

FIG. 2 is a back view of a Bomber Style Headwear embodiment of the present invention.

FIG. 3 is a side view of a Brimmed Summer Style Headwear embodiment of the present invention.

FIG. 4 is a back view of a Brimmed Summer Style Headwear embodiment of the present invention.

FIG. 5 is a side view of a Winter Pom Style Headwear embodiment of the present invention.

FIG. 6 is a back view of a Winter Pom Style Headwear embodiment of the present invention.

FIG. 7 is a front side view of a Baseball Cap Style Headwear embodiment of the present invention.

FIG. 8 is a back side view of a Baseball Cap Style Headwear embodiment of the present invention.

### DETAILED DESCRIPTION OF THE INVENTION

In the following description, reference is made to the accompanying drawings that form a part hereof, and in which is shown by way of illustration, specific embodiments which may be practiced. These embodiments are described in sufficient detail to enable those skilled in the art to practice the invention, and it is to be understood that other embodiments may be utilized, and that structural, mechanical, logical and electrical changes may be made without departing from the scope of the present invention. The following description of example embodiments is, therefore, not to be taken in a limited sense, and the scope of the present invention is defined by the appended claims.

The present invention is directed to headwear items adapted to receive cochlear implant (“CI”) transmitters such that the CI transmitters are maintained in the appropriate position to interact and function with a cochlear implant receiver. Specifically, headwear items of the present invention utilize an access point to allow insertion of a CI transmitter under an external layer of the headwear and positioning of the CI transmitter to allow magnetic adhesion with the corresponding internal components of the cochlear implant. Exemplar embodiments of headwear of the present invention and methods of use are described in more detail herein. As used herein, the term “CI transmitters” refers to CI transmitters and similar technology as known to one of skill in the art.

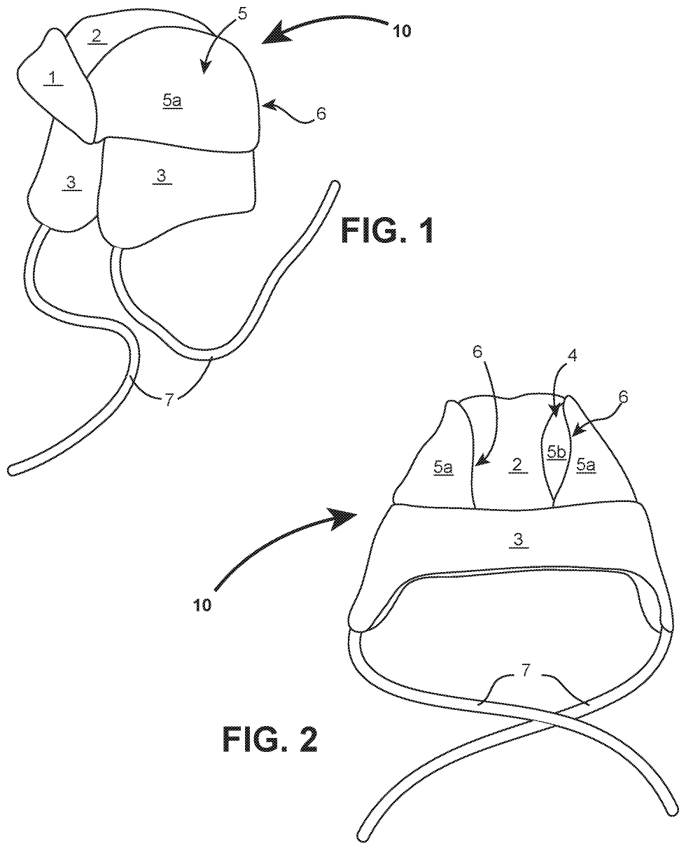

Turning to FIGS. 1 and 2, a Bomber Style Headwear 10 embodiment of the present invention is shown. The Bomber Style Headwear 10 includes one or more side panels 5, each side panel 5 comprising an external layer 5a and an internal layer 5b arranged such that the internal layer 5b and the external layer 5a are positioned and attached to create one or more pockets 4. Constructed of heavier, cold weather material such as wool or poly knit, fleece, woven or wind-barrier fiber, the external layer 5a of the one or more side panels 5 is attached to the internal layer 5b, using stitching, bonding, or similar methods known in the art, to create the one or more pockets 4 in which the CI transmitter may be ensconced. The internal layer 5b may be made of similar materials, but should be sufficiently thin to allow magnetic adhesion between the CI transmitter and the corresponding internal components of the cochlear implant. In other embodiments, the one or more side panels 5 may be constructed of alternative materials, including natural and synthetic fabrics known in the art to be suitable for hat construction.

One or more access points 6 are openings formed in each of the one or more side panels 5 to provide access to the one or more pockets 4 formed therein. The one or more access points 6 may be positioned at the rear of the one or more side panels 5 along the edge of the external layer 5a according to various embodiments. However, the position of the one or more access points 6 may vary along the edge of the external layer 5a or may be formed by creating a slot in the external layer 5a. Alternatively, the position of the one or more access points 6 may be formed by creating a slot in the internal layer 5b. According to some embodiments, the one or more access points 6 may include closure solutions, such as friction, hook-and-loop fasteners, buttons, snaps, hook and eye, zippers, any other closure solution known in the art, or any combination thereof. In other embodiments, no closure solutions are featured. For example, portions of the external layer 5a on either side of a slot may be overlapped to create the one or more access points 6.

As shown, the Bomber Style Headwear 10 may include one or more ear flaps 3 attached to and extending below the one or more side panels 5 to provide additional protection for the wearer's ears according to various embodiments. In addition, the Bomber Style Headwear 10 may include a top panel 2, connecting the one or more side panels 5. One or more neck ties 7 may be attached to and extend from the one or more ear flaps 3 to secure the Bomber Style Headwear 10 when worn by the user. According to some embodiments, the one or more neck ties 7 may include ties, straps, clips, any other neck tie known in the art, or any combination thereof.

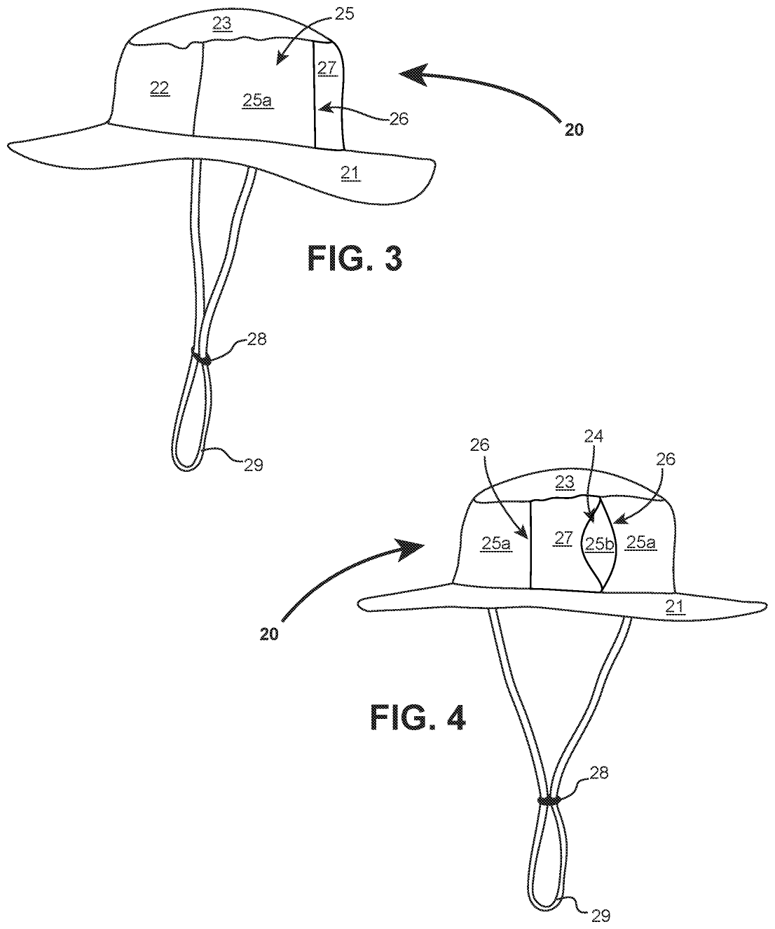

Turning to FIGS. 3 and 4, a Brimmed Summer Style Headwear 20 embodiment of the present invention is shown. The Brimmed Summer Style Headwear 20 includes one or more side panels 25, each side panel 25 comprising an external layer 25a and an internal layer 25b arranged such that the internal layer 25b and the external layer 25a are positioned and attached to create one or more pockets 24. Constructed of thinner, warm weather material such as natural or synthetic knit, woven or wind-barrier fiber, the external layer 25a of the one or more side panels 25 is attached to the internal layer 25b, using stitching, bonding, or similar methods known in the art, to create the one or more pockets 24 in which the CI transmitter may be ensconced. The internal layer 25b may be made of similar materials, but should be sufficiently thin to allow magnetic adhesion between the CI transmitter and the corresponding internal receiver components of the cochlear implant. In other embodiments, the one or more side panels 25 may be constructed of alternative materials, including natural and synthetic fabrics known in the art to be suitable for hat construction.

One or more access points 26 are openings formed in each of the one or more side panels 25 to provide access to the one or more pockets 24 formed therein. The one or more access points 26 may be positioned at the rear of the one or more side panels 25 along the edge of the external layer 25a according to various embodiments. However, the position of the one or more access points 26 may vary along the edge of the external layer 25a or may be formed by creating a slot in the external layer 25a. Alternatively, the position of the one or more access points 26 may be formed by creating a slot in the internal layer 25b. According to some embodiments, the one or more access points 26 may include closure solutions, such as friction, hook-and-loop fasteners, buttons, snaps, hook and eye, zippers, any other closure solution known in the art, or any combination thereof. In other embodiments, no closure solutions are featured. For example, portions of the external layer 25a on either side of a slot may be overlapped to create the one or more access points 26.

As shown, the Brimmed Summer Style Headwear 20 may include a brim 21 attached to and extending from the one or more side panels 25 to protect the wearer from sun exposure according to various embodiments. In addition, the Brimmed Summer Style Headwear 20 may include a front passive panel 22, a top panel 23 and a back passive panel 27, connecting the one or more side panels 25. One or more neck ties 29 may be attached to and extend from either the one or more side panels 25 or the brim 21 to secure the Brimmed Summer Style Headwear 20 when worn by the user. According to some embodiments, the one or more neck ties 29 may include ties, straps, clips, any other neck tie known in the art, or any combination thereof. Further, an adjustable slider 28 may be mated with the one or more neck ties 29 for adjusting the fit of the Brimmed Summer Style Headwear 20.

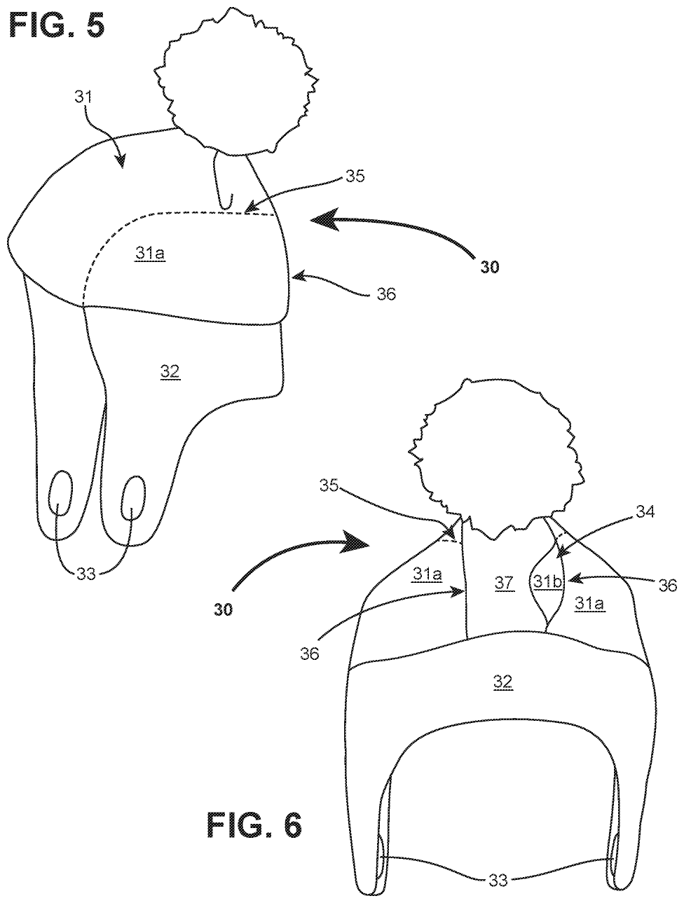

Turning to FIGS. 5 and 6, a Winter Pom Style Headwear 30 embodiment of the present invention is shown. The Winter Pom Style Headwear 30 includes a body panel comprising one or more side panels 31, each side panel 31 comprising an external layer 31a and an internal layer 31b arranged such that the internal layer 31b and the external layer 31a are positioned and attached to create one or more pockets 34. Constructed of heavier, cold weather material such as wool or poly knit, fleece, woven or wind-barrier fiber, the external layer 31a of the one or more side panels 31 is attached to the internal layer 31b, using stitching, bonding, or similar methods known in the art, to create the one or more pockets 34 in which the CI transmitter may be ensconced. As depicted, an interior pocket division 35 is created with stitching that defines the one or more pockets 34. The internal layer 31b may be made of similar materials, but should be sufficiently thin to allow magnetic adhesion between the CI transmitter and the corresponding internal receiver components of the cochlear implant. In other embodiments, the one or more side panels 31 may be constructed of alternative materials, including natural and synthetic fabrics known in the art to be suitable for hat construction.

One or more access points 36 are openings formed in each of the one or more side panels 31 to provide access to the one or more pockets 34 formed therein. The one or more access points 36 may be positioned at the rear of the one or more side panels 31 along the edge of the external layer 31a according to various embodiments. However, the position of the one or more access points 36 may vary along the edge of the external layer 31a or may be formed by creating a slot in the external layer 31a. Alternatively, the position of the one or more access points 36 may be formed by creating a slot in the internal layer 31b. According to some embodiments, the one or more access points 36 may include closure solutions, such as friction, hook-and-loop fasteners, buttons, snaps, hook and eye, zippers, any other closure solution known in the art, or any combination thereof. In other embodiments, no closure solutions are featured. For example, portions of the external layer 31a on either side of a slot may be overlapped to create the access point 36.

As shown, the Winter Pom Style Headwear 30 may include one or more ear flaps 32 attached to and extending from the one or more side panels 31 to provide additional protection for the wearer's ears according to various embodiments. In addition, the Winter Pom Style Headwear 30 may include a back passive panel 37, connecting the one or more side panels 31. One or more neck strap closures 33 may be included to secure the Winter Porn Style Headwear 30 when worn by the user. According to some embodiments, the one or more neck strap closures 33 may include friction, Hook-and-loop fasteners, button, snap, hook and eye, zipper, any other neck strap closures known in the art, or any combination thereof.

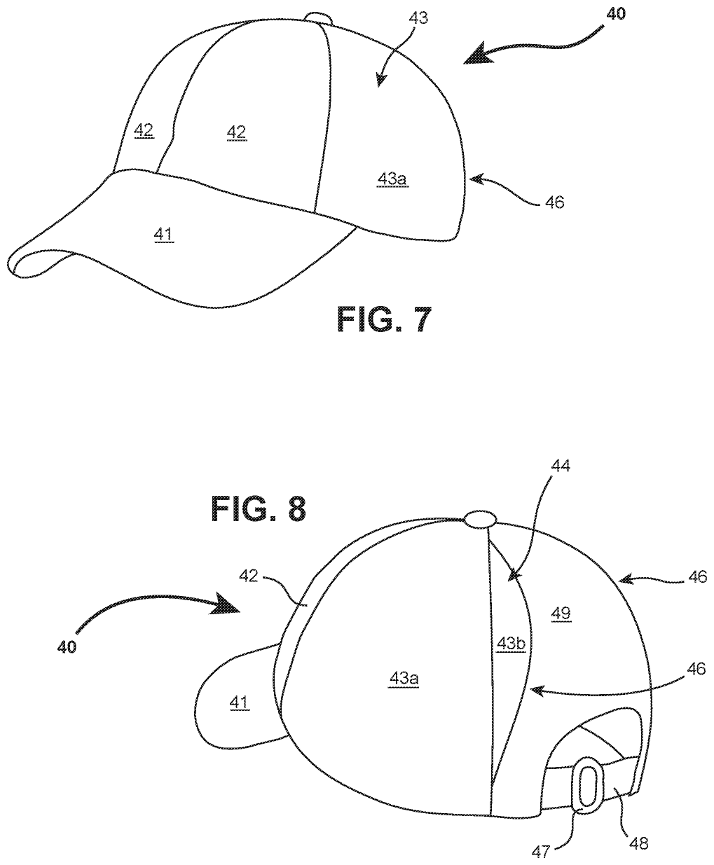

Turning to FIGS. 7 and 8, a Baseball Cap Style Headwear 40 embodiment of the present invention is shown. The Baseball Cap Style Headwear 40 includes one or more side panels 43, each side panel 43 comprising an external layer 43a and an internal layer 43b arranged such that the internal layer 43b and the external layer 43a are positioned and attached to create one or more pockets 44. Constructed of thinner, warm weather material such as natural or synthetic knit, woven or wind-barrier fiber, the external layer 43a of the one or more side panels 43 is attached to the internal layer 43b, using stitching, bonding, or similar methods known in the art, to create the one or more pockets 44 in which the CI transmitter may be ensconced. The internal layer 43b may be made of similar materials, but should be sufficiently thin to allow magnetic adhesion between the CI transmitter and the corresponding internal receiver components of the cochlear implant. In other embodiments, the one or more side panels 43 may be constructed of alternative materials, including natural and synthetic fabrics known in the art to be suitable for hat construction.

One or more access points 46 are openings formed in each of the one or more side panels 43 to provide access to the one or more pockets 44 formed therein. The one or more access points 46 may be positioned at the rear of the one or more side panels 43 along the edge of the external layer 43a according to various embodiments. However, the position of the one or more access points 46 may vary along the edge of the external layer 43a or may be formed by creating a slot in the external layer 43a. Alternatively, the position of the one or more access points 46 may be formed by creating a slot in the internal layer 43b. According to some embodiments, the one or more access points 46 may include closure solutions, such as friction, hook-and-loop fasteners, buttons, snaps, hook and eye, zippers, any other closure solution known in the art, or any combination thereof. In other embodiments, no closure solutions are featured. For example, portions of the external layer 43a on either side of a slot may be overlapped to create the access point 46.

As shown, the Baseball Cap Style Headwear 40 may include a brim 41 attached to and extending from the one or more side panels 43 to protect the wearer from sun exposure according to various embodiments. In addition, the Baseball Cap Style Headwear 40 may include one or more front passive panels 42. Further, the Baseball Cap Style Headwear 40 may include a back passive panel 49, connecting the one or more side panels 43. In exemplary embodiments, the one or more from passive panels 42 may be attached to and positioned between a pair of side panels 43 wherein the brim 41 is attached to and extends from the one or more passive front panels 42 and a portion of the pair of side panels 43. The Baseball Cap Style Headwear 40 may include an adjustable strap 48 and a buckle slider 47 for adjusting the fit of the Baseball Cap Style Headwear 40. The adjustable strap 48 and the buckle slider 47 may be any adjustable closure mechanism known in the art.

Turning to a first method of using the medical support headwear, a first step is providing a Bomber Style Headwear 10 embodiment including at least one or more side panels 5, each side panel 5 comprising an external layer 5a and an internal layer 5b arranged such that the internal layer 5b and the external layer 5a are positioned and attached to create one or more pockets 4, one or more access points 6, one or more ear flaps 3, and a top panel 2. Optionally, the Bomber Style Headwear 10 may include one or more access point closure solutions or one or more neck ties 7. A second step includes placing the Bomber Style Headwear 10 on the user's head. In embodiments of the method where the provided Bomber Style Headwear 10 includes one or more neck ties 7, the second step of placing the Bomber Style Headwear 10 on the user's head may optionally include securing the Bomber Style Headwear 10 with the one or more neck ties 7 once the Bomber Style Headwear 10 is placed on the user's head. A third step includes opening the access point 6 formed in a first of the one or more side panels 5 by separating the external layer 5a from the internal layer 5b to create an open access point 6. A fourth step includes inserting a CI transmitter through the open access point 6 and into the pocket 4 such that the CI transmitter lies in the pocket 4 and in magnetic adhesion with the cochlear implant. A fifth step includes closing the open access point 6 such that the CI transmitter is ensconced in the pocket 4. An optional sixth step includes adjusting the position of the CI transmitter once the Bomber Style Headwear 10 is fully secured. An optional seventh step in embodiments of the Bomber Style Headwear 10 where one or more access point closure solutions are provided includes securing the one or more access points 6 with the provided access point closure solutions. An optional eighth step includes repeating steps three through seven for the access point 6 formed in a second of the one or more side panels 5.

Turning to a second method of using the medical support headwear, a first step is providing a Brimmed Summer Style Headwear 20 embodiment including at least one or more side panels 25, each side panel 25 comprising an external layer 25a and an internal layer 25b arranged such that the internal layer 25b and the external layer 25a are positioned and attached to create one or more pockets 24, one or more access points 26, a top panel 23, a front passive panel 22, a back passive panel 27, and a brim 21. Optionally, the Brimmed Summer Style Headwear 20 may include one or more access point closure solutions, one or more neck ties 29 or an adjustable slider 28. A second step includes placing the Brimmed Summer Style Headwear 20 on the user's head. In embodiments of the method where the provided Brimmed Summer Style Headwear 20 includes one or more neck ties 29, the second step of placing the Brimmed Summer Style Headwear 20 on the user's head may optionally include securing the Brimmed Summer Style Headwear 20 with the one or more neck ties 29 once the Brimmed Summer Style Headwear 20 is placed on the user's head. Similarly, in embodiments of the Brimmed Summer Style Headwear 20 where an adjustable slider 28 is provided, the second step may optionally include adjusting the fit of the Brimmed Summer Style Headwear 20 with the adjustable slider 28. A third step includes opening the access point 26 formed in a first of the one or more side panels 25 by separating the external layer 25a from the internal layer 25b to create an open access point 26. A fourth step includes inserting a CI transmitter through the open access point 26 and into the pocket 24 such that the CI transmitter lies in the pocket 24 and in magnetic adhesion with the cochlear implant. A fifth step includes closing the open access point 26 such that the CI transmitter is ensconced in the pocket 24. An optional sixth step includes adjusting the position of the CI transmitter once the Brimmed Summer Style Headwear 20 is fully secured. An optional seventh step in embodiments of the Brimmed Summer Style Headwear 20 where one or more access point closure solutions are provided includes securing the one or more access points 26 with the provided access point closure solutions. An optional eighth step includes repeating steps three through seven for the access point 26 formed in a second of the one or more side panels 25.

Turning to a third method of using the medical support headwear, a first step is providing a Winter Pom Style Headwear 30 embodiment including at least one or more side panels 31, each side panel 31 comprising an external layer 31a and an internal layer 31b arranged such that the internal layer 31b and the external layer 31a are positioned and attached to create one or more pockets 34, one or more interior pocket divisions 35, one or more access points 36, a back passive panel 37, and one or more ear flaps 32. Optionally, the Winter Pom Style Headwear 30 may include one or more access point closure solutions or one or more neck strap closures 33. A second step includes placing the Winter Pom Style Headwear 30 on the user's head. In embodiments of the method where the provided Winter Pom Style Headwear 30 includes one or more neck strap closures 33, the second step of placing the Winter Pom Style Headwear 30 on the user's head may optionally include securing the Winter Pom Style Headwear 30 with the one or more neck strap closures 33 once the Winter Pom Style Headwear 30 is placed on the user's head. A third step includes opening the access point 36 formed in a first of the one or more side panels 31 by separating the external layer 31a from the internal layer 31b to create an open access point 36. A fourth step includes inserting a CI transmitter through the open access point 36 and into the pocket 34 such that the CI transmitter lies in the pocket 34 and in magnetic adhesion with the cochlear implant. A fifth step includes closing the open access point 36 such that the CI transmitter is ensconced in the pocket 34. An optional sixth step includes adjusting the position of the CI transmitter once the Winter Pom Style Headwear 30 is fully secured. An optional seventh step in embodiments of the Winter Pom Style Headwear 30 where one or more access point closure solutions are provided includes securing the one or more access points 36 with the provided access point closure solutions. An optional eighth step includes repeating steps three through seven for the access point 36 formed in a second of the one or more side panels 31.

Turning to a fourth method of using the medical support headwear, a first step is providing a Baseball Cap Style Headwear 40 embodiment including at least one or more side panels 43, each side panel 43 comprising an external layer 43a and an internal layer 43b arranged such that the internal layer 43b and the external layer 43a are positioned and attached to create one or more pockets 44, one or more access points 46, a brim 41, one or more front passive panels 42, and a back passive panel 49. Optionally, the Baseball Cap Style Headwear 40 may include one or more access point closure solutions, one or more adjustable straps 48, or a buckle slider 47. A second step includes placing the Baseball Cap Style Headwear 40 on the user's head. In embodiments of the method where the provided Baseball Cap Style Headwear 40 includes one or more adjustable straps 48, the second step of placing the Baseball Cap Style Headwear 40 on the user's head may optionally include adjusting the fit of the Baseball Cap Style Headwear 40 with the one or more adjustable straps 48 once the Baseball Cap Style Headwear 40 is placed on the user's head. A third step includes opening the access point 46 formed in a first of the one or more side panels 43 by separating the external layer 43a from the internal layer 43b to create an open access point 46. A fourth step includes inserting a CI transmitter through the open access point 46 and into the pocket 44 such that the CI transmitter lies in the pocket 44 and in magnetic adhesion with the cochlear implant. A fifth step includes closing the open access point 46 such that the CI transmitter is ensconced in the pocket 44. An optional sixth step includes adjusting the position of the CI transmitter once the Baseball Cap Style Headwear 40 is fully secured. An optional seventh step in embodiments of the Baseball Cap Style Headwear 40 where one or more access point closure solutions are provided includes securing the one or more access points 46 with the provided access point closure solutions. An optional eighth step includes repeating steps three through seven for the access point 46 formed in a second of the one or more side panels 43.

It is noted that the foregoing examples have been provided merely for the purpose of explanation and are in no way to be construed as limiting of the present invention. While the present invention has been described with reference to exemplary embodiments, it is understood that the words, which have been used herein, are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A medical support headwear device comprising:
one or more side panels;
each of the one or more side panels comprising an internal layer attached to an external layer to form a pocket therein; and
an access point formed in each of the one or more side panels to provide access to the pocket formed therein.

2. The medical support headwear device of claim 1 wherein the external layer of the one or more side panels has an edge and the access point is located along the edge of the external layer.

3. The medical support headwear device of claim 1 wherein the access point is a slot in the external layer of the side panel.

4. The medical support headwear device of claim 1 further comprising ear flaps attached to and extending below the one or more side panels and one or more neck ties attached to and extending from the ear flaps.

5. The medical support headwear device of claim 1 further comprising a brim attached to and extending from the one or more side panels.

6. The medical support headwear device of claim 5 further comprising one or more neck ties and an adjustable slider, the adjustable slider mated with the one or more neck ties and the one or more neck ties attached to and extending from either the one or more side panels or the brim.

7. The medical support headwear device of claim 5 further comprising one or more front passive panels attached to and positioned between a pair of side panels wherein the brim is attached to and extends from the one or more passive front panels and a portion of the pair of side panels.

8. The medical support headwear device of claim 1 wherein the one or more side panels is a body panel having an internal and external layer and the internal layer and external layer are attached by an interior pocket division to form one or more pockets.

9. The medical support headwear device of claim 8 further comprising ear flaps attached to and extending from the body panel.

10. A method for wearing medical support headwear comprising:
a. providing a medical support headwear device comprising:
one or more side panels;
each of the one or more side panels comprising an internal layer attached to an external layer to form a pocket therein; and
an access point formed in each of the one or more side panels to provide access to the pocket formed therein;
b. placing the medical support headwear device on the user's head;
c. opening the access point formed in a first of the one or more side panels by separating the external layer from the internal layer to create an open access point;
d. inserting a CI transmitter through the open access point and into the pocket such that the CI transmitter lies in the pocket and in magnetic adhesion with the corresponding internal receiver components of the cochlear implant; and
e. closing the open access point such that the CI transmitter is ensconced in the pocket.

11. The method of claim 10 further comprising adjusting the position of the CI transmitter ensconced in the pocket of the first of the one or more side panels.

12. The method of claim 11 further comprising repeating steps c through e for the access point formed in a second of the one or more side panels.

13. The method of claim 12 further comprising adjusting the position of the CI transmitter ensconced in the pocket of the second of the one or more side panels.
